(19) United States
(12) Patent Application Publication
(10) Pub. No.: US 2025/0380911 A1
(43) Pub. Date: Dec. 18, 2025

(54) METHOD TO ESTABLISH VITAL SIGN PREDICTION MODELS AND APPLICATIONS THEREOF

(71) Applicant: NATIONAL TAIWAN UNIVERSITY HOSPITAL, Taipei (TW)

(72) Inventor: CHIEN-CHANG LEE, Taipei (TW)

(21) Appl. No.: 18/743,150

(22) Filed: Jun. 14, 2024

(51) Int. Cl. A61B 5/00; A61B 5/0205; A61B 5/024; A61B 5/145; G16H 50/20

(52) U.S. Cl. CPC A61B 5/7275; A61B 5/0205; A61B 5/7267; G16H 50/20; A61B 5/024; A61B 5/14542

(57) ABSTRACT

The present invention relates to a method to establish vital sign prediction models to predict future vital signs of a patient. The method comprises training an attention-based architecture with a vital sign training dataset comprising multiple vital sign training data, each of which has a training input and a training ground truth, wherein the multiple training data comprise multiple control training data of multiple first patients without critical conditions and multiple emergency training data of multiple second patients with critical conditions. The present invention also provides an application of the established vital sign prediction models to realize an early warning system to predict cardiac arrest at earlier time points.

### FIELD OF THE INVENTION

The present invention relates to a prediction system to predict future vital signs of a patient, which enables medical personnel to take necessary medical treatment in advance if critical conditions are forecasted.

### DESCRIPTION OF RELATED ART

In medical institutions, it is crucial to monitor high-risk patients to take necessary medical treatment in time and prevent avoidable deaths. Traditionally, scoring warning systems such as early warning score (EWS), which use weighted aggregation of vital signs, are applied to evaluate severity of a patient. Recently, AI has been used to more accurately predict severe outcomes such as cardiac arrest and respiratory failure.

However, most of the above prediction system focus on predicting specific outcomes of a patient without providing future trend of monitored vital signs. Compared to the prediction result alone, the trend of vital signs may provide more information for medical personnel to choose appropriate treatment. The method and system to predict future trend of various vital signs, however, is still not available up to date.

To improve the healthcare quality and reduce avoidable deaths for patients, there is a need to develop a vital sign prediction system to forecast various vital signs based on collected physiological data.

### SUMMARY OF THE INVENTION

The present invention relates to methods of establishing prediction models to predict future vital signs of a patient. The present invention also relates to a prediction system to evaluate the risk of sudden death by combining a plurality of vital sign prediction models and a cardiac arrest prediction model

In one aspect, the present invention provides a method for training a prediction model to predict future vital signs of a subject within a predetermined future time interval. The method comprises training an attention-based architecture with a vital sign training dataset comprising multiple vital sign training data, each of which has a training input and a training ground truth. Regarding training data, the multiple vital sign training data comprise (1) multiple control training data of multiple first patients without critical conditions, and (2) multiple emergency training data of multiple second patients with critical conditions. The training input comprises one or more static variables and time series data of a target variable and one or more time-dependent unknown variables in an observation window. The training ground truth comprises time series data of the target variable in a forecast window. In one preferred embodiment, the attention-based architecture is temporal fusion transformer.

In one embodiment, the training input further comprises time series data of one or more time-dependent known variables in the observation window and the forecast window. The time-dependent known variables may be daytime and nighttime.

In one embodiment, the multiple second patients have critical conditions occur in the observation window or the forecast window. The critical conditions may be cardiac arrest, shock, and/or respiratory failure. In another embodiment, some of the multiple second patients have critical conditions occur outside the observation window and the forecast window, and may only be found in original records of the second patients corresponding to the multiple vital sign training data. The critical conditions may be cardiac arrest, shock, respiratory failure, or other types of outcomes.

The one or more static variables may include but not limited to a comorbidity label, a BMI value, an oxygen supply status, and a state of consciousness. The target variable is a vital sign selected from a group of vital signs. In one embodiment, the group of vital signs consisting of heart rate, respiratory rate, systolic blood pressure, diastolic blood pressure, mean arterial pressure, and blood oxygen saturation. The remaining vital signs which are not selected are used as time-dependent unknown variables. Other vital signs such as body temperature or blood glucose level may also be used as time-dependent unknown variables in the model.

In one embodiment, the observation window comprises 24 consecutive past time points, and the forecast window comprises 12 consecutive future time points. In one preferred embodiment, the time interval between two consecutive time points is 5 minutes. In this case, the observation window is 2 hours, and the forecast window is 1 hour.

In another aspect of the present invention provides a method to predict future risk of sudden death of a subject. The method comprises the steps of (1) obtaining one or more static variables of the subject; (2) obtaining measured time series data of multiple vital signs from the subject; (3) based on the measured time series data and the multiple static variables, predicting forecasted time series data of heart rate by a heart rate prediction model, and predicting forecasted time series data of blood oxygen saturation by an oxygen saturation prediction model; and (4) based on the forecasted time series data of heart rate, the forecasted time series data of blood oxygen saturation, and/or the measured time series data, predicting a risk of sudden death of the subject by a cardiac arrest prediction model. The one or more static variables may include but not limited to a comorbidity label, a BMI value, an oxygen supply status, and a state of consciousness. The multiple vital signs at least contain heart rate and blood oxygen saturation data. In preferred embodiments, the multiple vital signs may further comprise respiratory rate, systolic blood pressure, diastolic blood pressure, and mean arterial pressure. The heart rate prediction model and the oxygen saturation prediction model are trained by the method for training a prediction model to predict future vital signs as described above. The cardiac arrest prediction model is trained by a cardiac arrest training dataset comprising multiple cardiac arrest training data, each of which consists essentially of time series data of heart rate, time series data of blood oxygen saturation, and a survival result of a patient.

In one embodiment, the survival result is a risk level or a probability of cardiac arrest of the subject.

In one embodiment, the forecasted time series data of heart rate and the forecasted time series data of blood oxygen saturation are predicted by using 2 hours of measured time series data and the one or more static variables to generate 1 hour of forecasted time series data. In one embodiment, the cardiac arrest prediction model uses 23 hours of measured time series data and 1 hour of forecasted time series data to predict the risk of sudden death

It should be understood that the above general descriptions and the following detailed descriptions are only for demonstrative and explanatory purposes, instead of limiting the scope of the present invention.

### BRIEF DESCRIPTION OF THE DRAWINGS

The above general descriptions and the following detailed descriptions of the present invention can be better understood with the figures. To illustrate the present invention, the preferred embodiments are provided in the figures. However, it should be understood that the present invention is not limited to the embodiments disclosed in the figures.

FIG. 1 shows the relation of training inputs and the training ground truth in a piece of training data.

FIG. 2 shows the architecture of a combined model with multiple vital sign prediction models and a cardiac arrest prediction model to predict a future risk of sudden death.

FIG. 3 shows that a 24-hour monitoring record of a patient is used to generate multiple pieces of training data.

FIGS. 4A-4F show the testing results for heart rate prediction model. FIG. 4A is the forecasted heart rate values vs. actual heart rate values in the testing data at the end of forecast window (τ=τₘₐₓ=12, which equals 1 hour). The mean absolute percentage error (MAPE) is also shown above. FIG. 4B shows one example of forecasted heart rate and the actual heart rate in the forecast window. The forecast intervals for different quantiles (0.02, 0.1, 0.25, 0.5, 0.75, 0.9 and 0.98) are also provided in the figure. FIG. 4C shows the attention of the vital signs at different time points of the observation window. FIG. 4D shows the importance of different static variables on heart rate forecasting. FIGS. 4E and 4F show the importance of different time-dependent variables on heart rate forecasting in encoder (FIG. 4E) and decoder (FIG. 4F).

FIGS. 5A-5F show the testing results for respiratory rate prediction model. FIG. 5A is the forecasted respiratory rate values vs. actual respiratory rate values in the testing data at the end of forecast window (τ=τₘₐₓ=12, which equals 1 hour). The mean absolute percentage error (MAPE) is also shown above. FIG. 5B shows one example of forecasted respiratory rate and the actual respiratory rate in the forecast window. The forecast intervals for different quantiles (0.02, 0.1, 0.25, 0.5, 0.75, 0.9 and 0.98) are also provided in the figure. FIG. 5C shows the attention of the vital signs at different time points of the observation window. FIG. 5D shows the importance of different static variables on respiratory rate forecasting. FIGS. 5E and 5F show the importance of different time-dependent variables on respiratory rate forecasting in encoder (FIG. 5E) and decoder (FIG. 5F).

FIGS. 6A-6F show the testing results for systolic blood pressure (SBP) prediction model. FIG. 6A is the forecasted SBP values vs. actual SBP values in the testing data at the end of forecast window (τ=τₘₐₓ=12, which equals 1 hour). The mean absolute percentage error (MAPE) is also shown above. FIG. 6B shows one example of forecasted SBP and the actual SBP in the forecast window. The forecast intervals for different quantiles (0.02, 0.1, 0.25, 0.5, 0.75, 0.9 and 0.98) are also provided in the figure. FIG. 6C shows the attention of the vital signs at different time points of the observation window. FIG. 6D shows the importance of different static variables on SBP forecasting. FIGS. 6E and 6F show the importance of different time-dependent variables on SBP forecasting in encoder (FIG. 6E) and decoder (FIG. 6F).

FIGS. 7A-7F show the testing results for diastolic blood pressure (DBP) prediction model. FIG. 7A is the forecasted DBP values vs. actual DBP values in the testing data at the end of forecast window (τ=τₘₐₓ=12, which equals 1 hour). The mean absolute percentage error (MAPE) is also shown above. FIG. 7B shows one example of forecasted DBP and the actual DBP in the forecast window. The forecast intervals for different quantiles (0.02, 0.1, 0.25, 0.5, 0.75, 0.9 and 0.98) are also provided in the figure. FIG. 7C shows the attention of the vital signs at different time points of the observation window. FIG. 7D shows the importance of different static variables on DBP forecasting. FIGS. 7E and 7F show the importance of different time-dependent variables on DBP forecasting in encoder (FIG. 7E) and decoder (FIG. 7F).

FIGS. 8A-8F show the testing results for mean arterial pressure (MAP) prediction model. FIG. 8A is the forecasted MAP values vs. actual MAP values in the testing data at the end of forecast window (τ=τₘₐₓ=12, which is equal to 1 hour). The mean absolute percentage error (MAPE) is also shown above. FIG. 8B shows one example of forecasted MAP and the actual MAP in the forecast window. The forecast intervals for different quantiles (0.02, 0.1, 0.25, 0.5, 0.75, 0.9 and 0.98) are also provided in the figure. FIG. 8C shows the attention of the vital signs at different time points of the observation window. FIG. 8D shows the importance of different static variables on MAP forecasting. FIGS. 8E and 8F show the importance of different time-dependent variables on MAP forecasting in encoder (FIG. 8E) and decoder (FIG. 8F).

FIGS. 9A-9F show the testing results for blood oxygen saturation (SpO₂) prediction model. FIG. 9A is the forecasted SpO₂ values vs. actual SpO₂ values in the testing data at the end of forecast window (τ=τₘₐₓ=12, which equals 1 hour). The mean absolute percentage error (MAPE) is also shown above. FIG. 9B shows one example of forecasted SpO₂ and the actual SpO₂ in the forecast window. The forecast intervals for different quantiles (0.02, 0.1, 0.25, 0.5, 0.75, 0.9 and 0.98) are also provided in the figure. FIG. 9C shows the attention of the vital signs at different time points of the observation window. FIG. 9D shows the importance of different static variables on SpO₂ forecasting. FIGS. 9E and 9F show the importance of different time-dependent variables on SpO₂ forecasting in encoder (FIG. 9E) and decoder (FIG. 9F).

FIG. 10 is the network architecture of LSTM training in this invention.

FIG. 11 is the network architecture of TCN training in this invention.

FIG. 12 shows the architecture of a combined model with a heart rate prediction model, a blood oxygen prediction model, and a cardiac arrest prediction model to predict a future risk of sudden death.

### DETAILED DESCRIPTION OF PREFERRED EMBODIMENTS

The following examples are referred to clearly explain the technical content, features and effects of this invention. Through the explanation of specific embodiments, a skilled artisan can further understand the technical means and effects adopted in the present invention to achieve the aforementioned purpose of the invention. In addition, the technology disclosed in this specification can be understood and implemented by a skilled artisan. Any changes or improvements consistent with the inventive concept are within the scope of the claims.

All the technical and scientific terms described in this specification and the claims, unless otherwise defined, are all with the definitions known by a person with ordinarily skilled in the art of this invention. A singular article “one”, “a”, “an”, “the”, or its approximate term, unless otherwise specified, can refer to more than one subject. The terms “or”, “and” used in the specification, unless otherwise specified, all refer to “and/or”. In addition, the terms “comprise” and “include” are open-ended terms which are not limiting. The aforementioned definition only describes the directed definition of the terms and should not be interpreted as a limitation to the claimed invention. Unless otherwise specified, the materials used in the present invention are all commercially available.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

The aim of the present invention is to provide a method to train an AI model to generate predicted future vital signs of a patient. The trained AI model is able to generate predicted vital signs within a predetermined future time interval. More specifically, temporal fusion transformer (TFT) architecture is used to construct the AI model.

In a journal article titled “Temporal Fusion Transformers for interpretable multi-horizon time series forecasting” (International Journal of Forecasting, 37(4), 1748-1764), Lim et al. described a temporal fusion transformers (TFT) having the features of (1) gating mechanisms to skip over any unused components of the architecture, (2) variable selection networks to select relevant input variables at each time step, (3) static covariate encoders to integrate static features into the network, (4) temporal processing to learn both long- and short-term temporal relationships from both observed and known time-varying inputs, and (5) prediction intervals via quantile forecasts to determine the range of likely target values at each prediction horizon.

As described by Lim et al., TFT architecture has quantile regression to multi-horizon forecasting setting, and may output different percentiles at each predicted time step. Each quantile forecast takes the form of ŷᵢ(q, t, τ)=f_{q}(τ, y_{i,t-k:t}, z_{i,t-k:t}, x_{i,t-k:t+τ}, sᵢ), where ŷᵢ(q, t, τ) is the predicted q^{th} sample quantile of the τ-step-ahead forecast at forecast start time t, and f_{q} is a prediction model which may use the input to generate corresponding predicted result. Here τ∈{1, . . . , τₘₐₓ}, where τₘₐₓ is forecast window, which is the length of time interval the model provides forecast results. In the model input part, k is observation window, which is the length of time interval the model uses to generate the forecast results. sᵢ are static covariates which are not altered during the whole time series. z_{i,t-k:t}, are unknown time-dependent time series data where the values of which in only known in the observation window and remain unknown in the forecast window. x_{i,t-k:t+τ} are known time-dependent time series data where the values of which in both the observation window and the forecast window can be known beforehand. y_{i,t-k:t} is the time series data of target feature in the observation window.

To predict ŷᵢ values in whole the observation window, the values of static covariates sᵢ, the observation window k, the forecast window τₘₐₓ, the values of target yᵢ from time point t−k to t, the values of unknown time-dependent inputs zᵢ from time point t−k to t, and the values of known time-dependent inputs xᵢ from time point t−k to t+τₘₐₓ are required. Thus, to construct training dataset for the model f_{q}, the above values are used as training input, and the actual values of target yᵢ in the forecast window (from time point t to t+τₘₐₓ) are used as corresponding training ground truths.

In the present invention, to establish a vital sign prediction model, a medical dataset comprising records of multiple patients is used to construct training datasets for model training. Each piece of data in the medical dataset comprises time series record of multiple vital signs and medical information of a patient. The multiple vital signs measure the body's most basic functions, which may include body temperature, heart rate, respiratory rate, systolic blood pressure, diastolic blood pressure, mean arterial pressure, and blood oxygen saturation. One of the multiple vital signs is selected as the target to forecast, and the other unselected vital signs are unknown time-dependent inputs in the training data. Medical information may include some information on medical record, such as height, weight, comorbidity, and state of consciousness. Most of such records are static covariates in the model. Besides, some other information may be derived from the multiple vital signs and/or the medical information, such as BMI values, daytime and nighttime. Depending on how they derived from, the values may be static covariates (such as BMI, which is derived from static covariates height and weight), known time-dependent inputs (such as daytime/nighttime, which is derived from location, date, and time), or unknown time-dependent inputs.

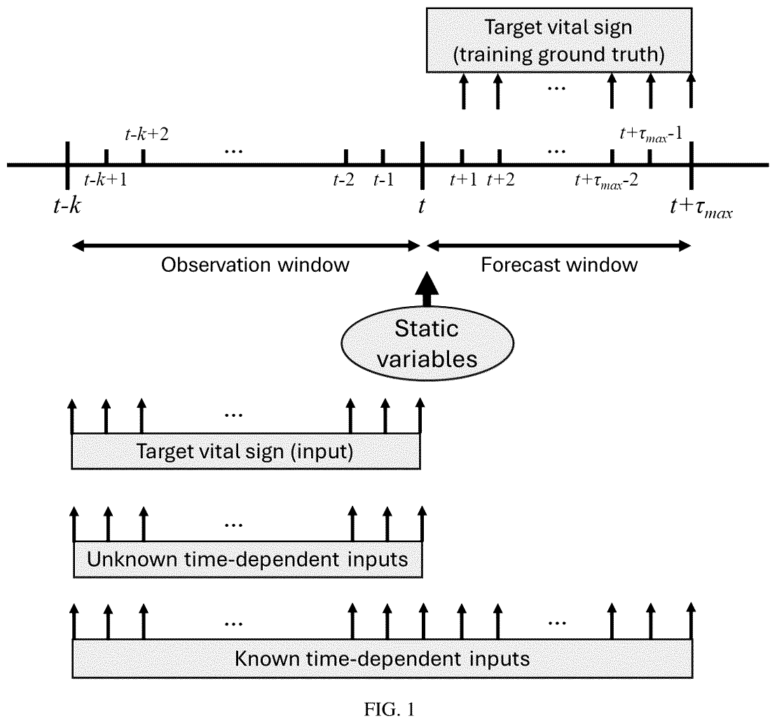

To construct a training dataset, one of the multiple vital signs is selected as the target of the prediction model. An observation window k and a forecast window τₘₐₓ are predetermined. As described above, the observation window ends at a forecast start point t, and the prediction window starts from the forecast start point. In each piece of data, a forecast start point is determined. Then the target vital sign and the unknown time-dependent inputs in observation window, the known time-dependent inputs in both observation window and forecast window, and the static covariates are combined to be a piece of training data. The target vital sign in the forecast window is set to be the corresponding training ground truth, as shown in FIG. 1.

Repeat the above step for multiple pieces of time series records in the medical dataset, a training dataset with multiple pieces of training data and corresponding training ground truths can be constructed, and can be used to train a prediction model to forecast the future trend of the target vital sign.

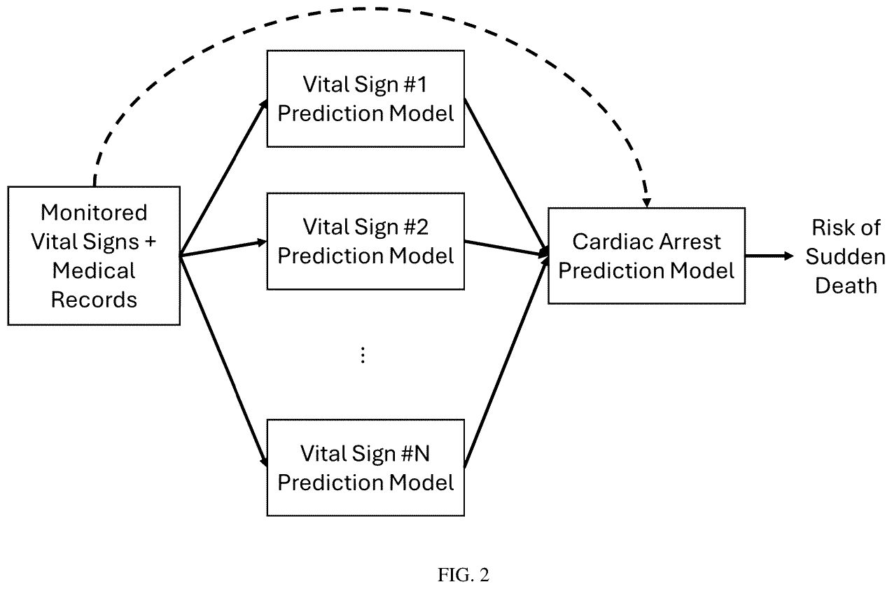

Besides, the trained vital sign prediction model may further be combined with other models. For example, a cardiac arrest prediction model may use the forecast results generated by various vital sign prediction models to predict a future risk of sudden death at an even earlier time point, as shown in FIG. 2.

### Example 1: Training Data Construction for Vital Sign Prediction Models

8,476 patient data from eICU Collaborative Research Database are used to construct the training dataset. Among those data, 677 patients are with cardiac arrest, 1,070 patients with shock, 1,668 patients with respiratory failure, and 5,061 patients without above conditions (control group). 6 vital signs are recorded in the patient data. Those vital signs are heart rate, respiratory rate, systolic blood pressure, diastolic blood pressure, mean arterial pressure, and blood oxygen saturation. In each training dataset, one of the 6 vital signs is selected as the target vital sign, and the other 5 vital signs are used as unknown time-dependent inputs. The age of patients, BMI level (determined by BMI value), oxygen supply condition, state of consciousness, and comorbidity label(s) are used as static covariates. The comorbidity labels are listed in the following Table:

| Number | Comorbidity |
|---|---|
| 1 | Congestive heart failure |
| 2 | Arrhythmia |
| 3 | Valvular disease |
| 4 | Pulmonary circulation disorder |
| 5 | Peripheral vascular disorder |
| 6 | Hypertension, uncomplicated |
| 7 | Hypertension, complicated |
| 8 | Paralysis |
| 9 | Other neurological dsiorder |
| 10 | Chronic pulmonary disease |
| 11 | Diabetes without chronic |
| | complications |
| 12 | Diabetes with chronic |
| | complications |
| 13 | Hypothyroidism |
| 14 | Renal failure |
| 15 | Liver disease |
| 16 | Chronic Peptic ulcer disease |
| | (includes bleeding only if |
| | obstruction is also present) |
| 17 | HIV and AIDS |
| 18 | Lymphoma |
| 19 | Metastatic cancer |
| 20 | Solid tumor without metastasis |
| 21 | Rheumatoid arthritis/collagen |
| | vascular diseases |
| 22 | Coagulopathy |
| 23 | Obesity |
| 24 | Weight loss |
| 25 | Fluid and electrolyte disorders |
| 26 | Blood loss anemia |
| 27 | Deficiency anemias |
| 28 | Alcohol abuse |
| 29 | Drug abuse |
| 30 | Psychoses |
| 31 | Depression |

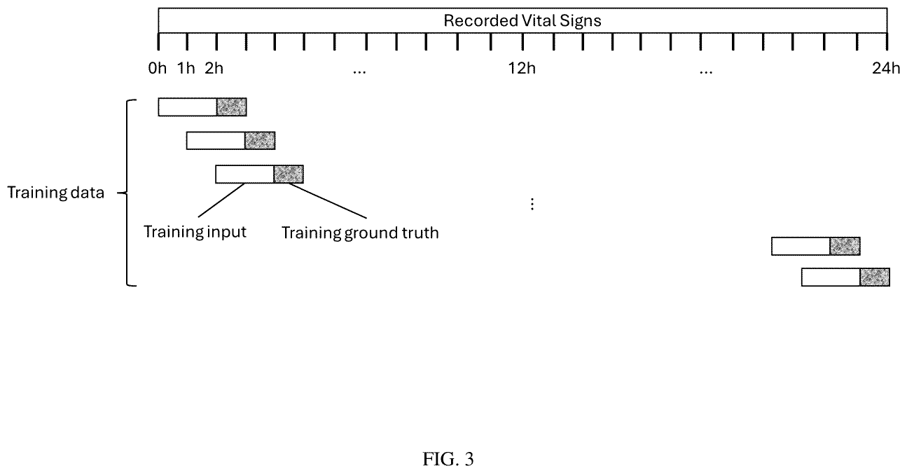

In eICU database, the multiple vital signs are recorded every 5 minutes for 24 hours. The observation window is set as 24 consecutive time points (2 hours), and the forecast window is set as 12 consecutive time points (1 hour). For each time series record in the database, there are 288 time points in total, and the record of a patient is used to generate multiple pieces of training data to forecast a specific vital sign, as shown in FIG. 3.

More specifically, in each of the above time series record, time points 1 to 252 are individually selected as the starting points of the observation windows in training data, with time points 25 to 276 become the starting points of corresponding forecast windows. The data of a selected starting point and 23 following time points are training input of a piece of training data, and the data of 12 time points after training input are the training ground truth of the training data. For example, data of time points 1 to 24 are used as the first training data, and data of time points 25 to 36 are used as corresponding first training ground truth. Data of time points 253 to 276 are used as input of a piece of testing data, and data of time points 277 to 288 are used as ground truth of the same piece of testing data. As such, 252 pieces of training data and 1 piece of testing data may be generated from each time series record. For all 5,061 included patients, a training set with 2,135,952 training data and a testing set with 8,476 testing data can be constructed.

### Example 2: Vital Sign Prediction Model Establishment and Model Efficacy

The 6 constructed datasets (one for each of the vital signs) are used to establish the vital sign prediction models. During training of each model, the maximum batch size is set as 50, with maximum training epochs of 200 and learning rate of 0.001, and the dropout rate is set as 0.1. The loss calculate takes quantile loss with quantile values of 0.02, 0.25, 0.5, 0.75, 0.9 and 0.98. The parameters are listed below:

$$limit\_train \_batches = 50 ,$$

$$max\_epochs = 200 ,$$

$$learning\_rate = 0.001 ,$$

$$loss = QuantileLoss ( ) ,$$

$$log\_interval = 10 ,$$

$$optimizer = Adam ,$$

$$reduce\_on \_plateau \_patience = 10 ,$$

$$early\_stop \_callback = EarlyStopping ( monitor ) = val_loss , \\ min\_delta = 1 e - 4 , patience = 10 , verbose = False , mode = min )$$

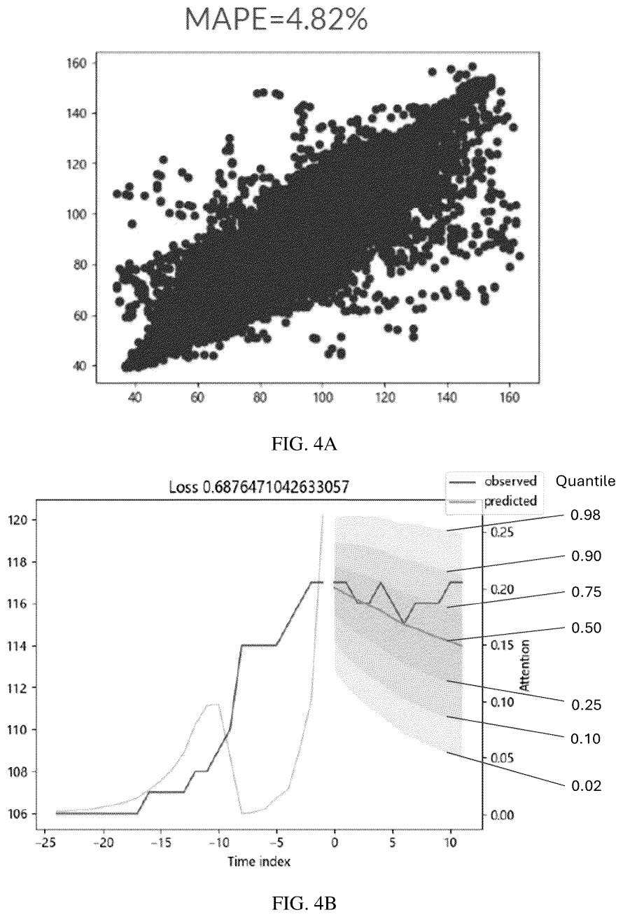

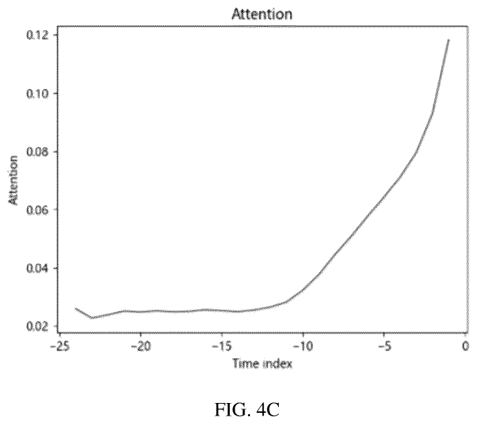

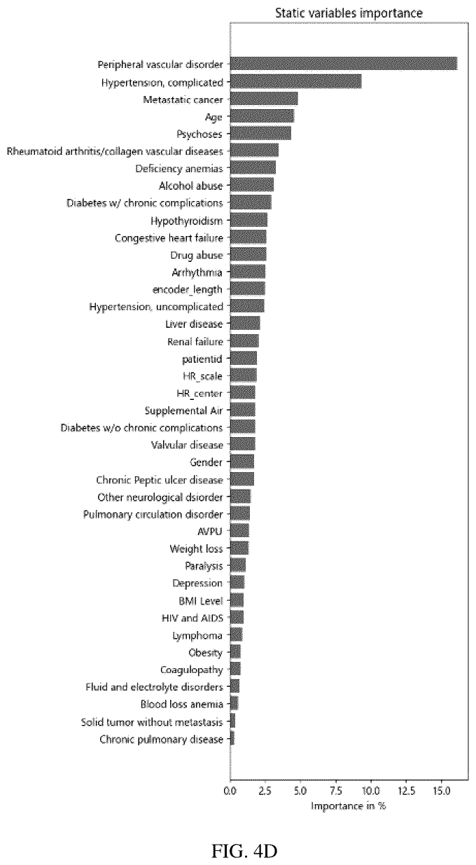

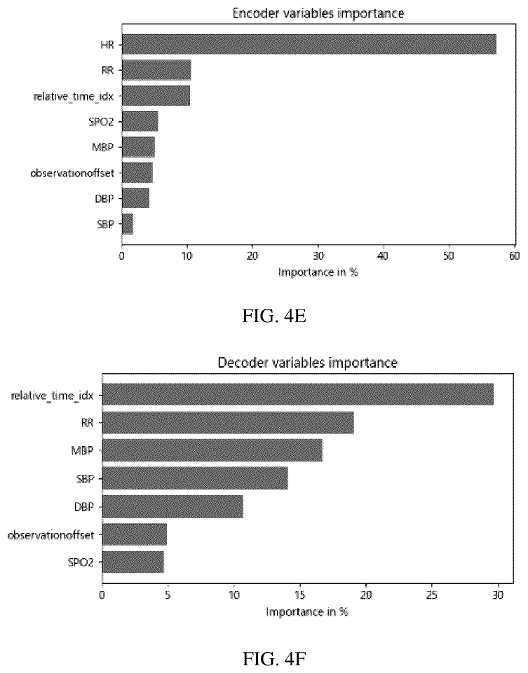

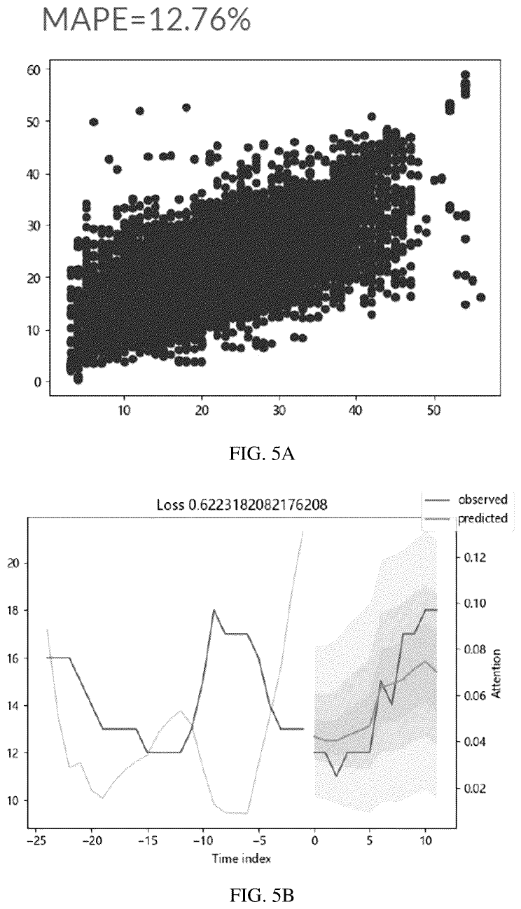

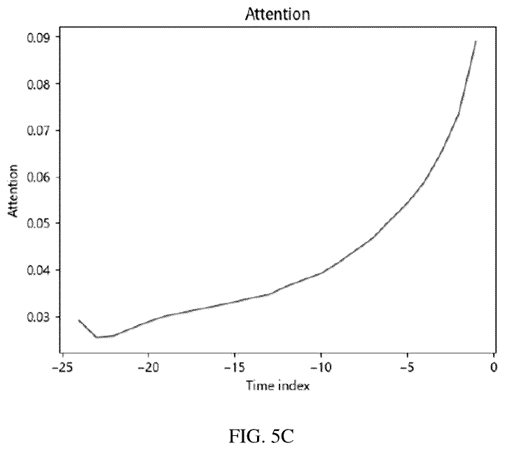

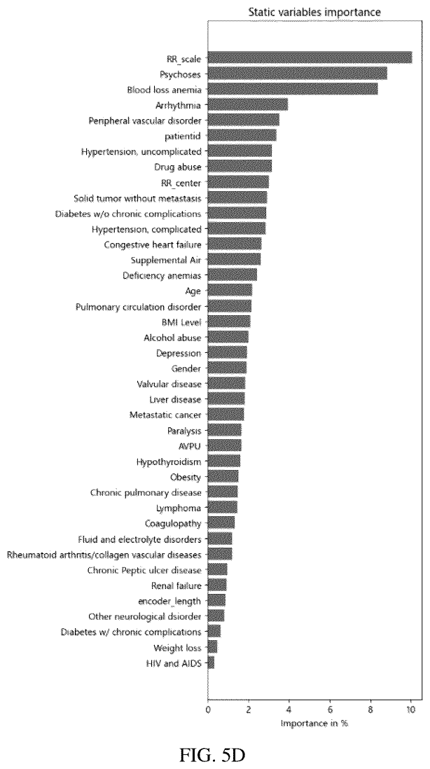

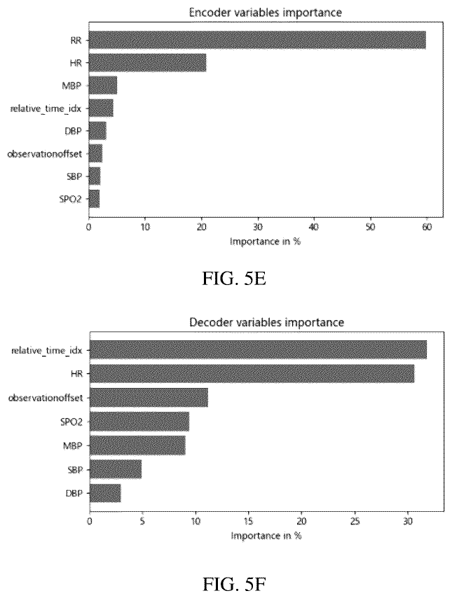

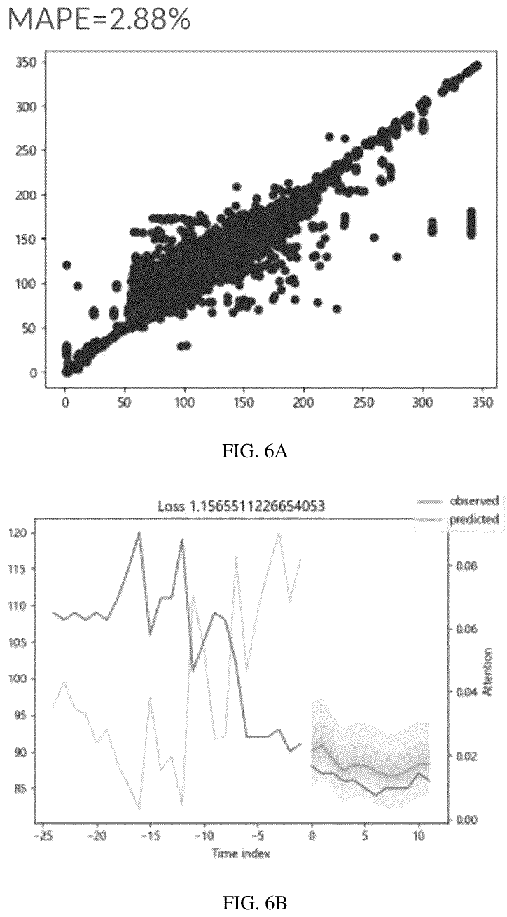

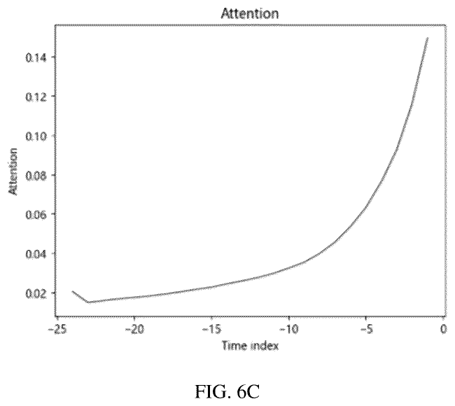

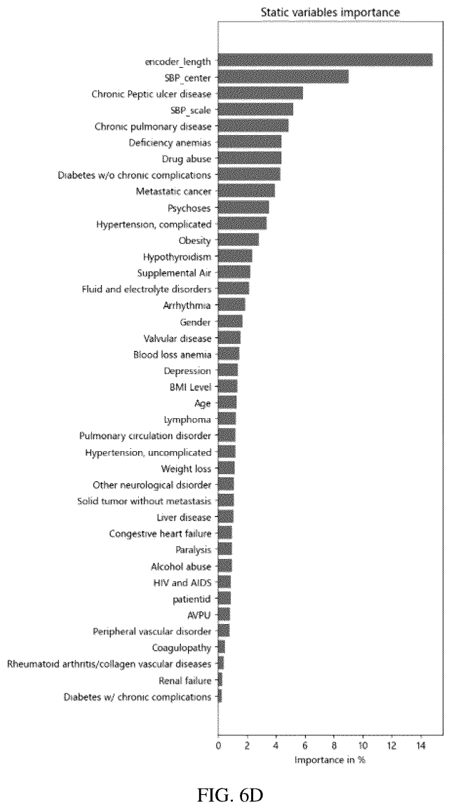

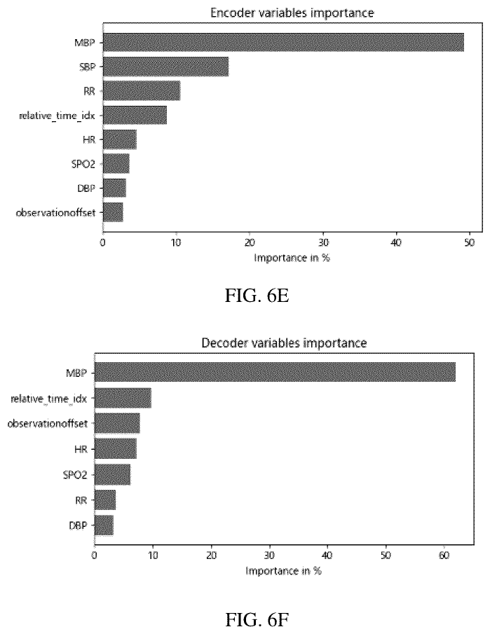

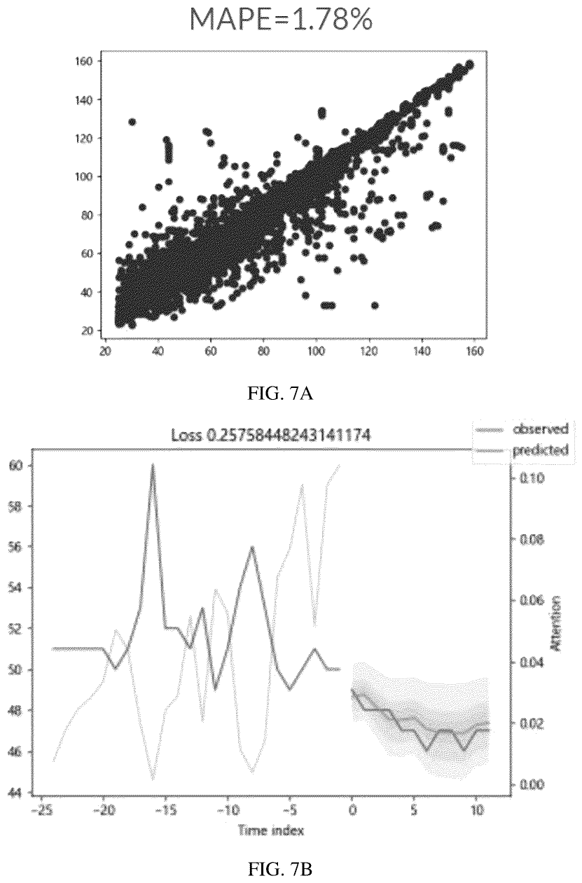

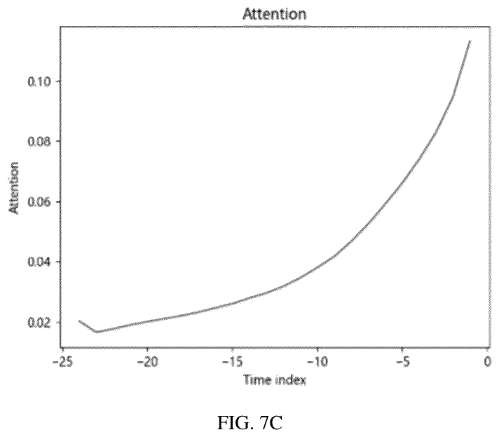

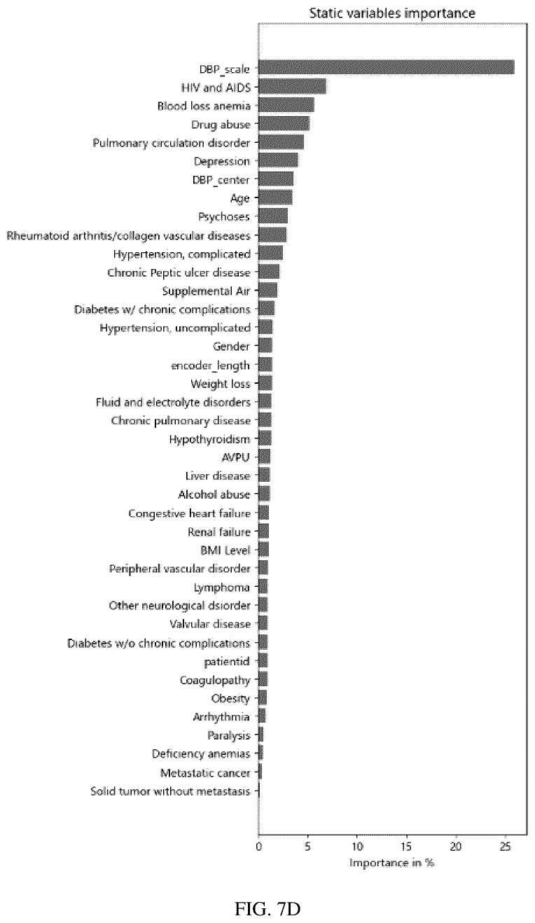

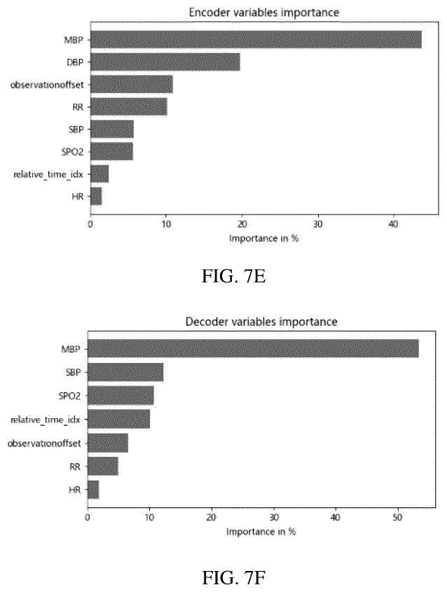

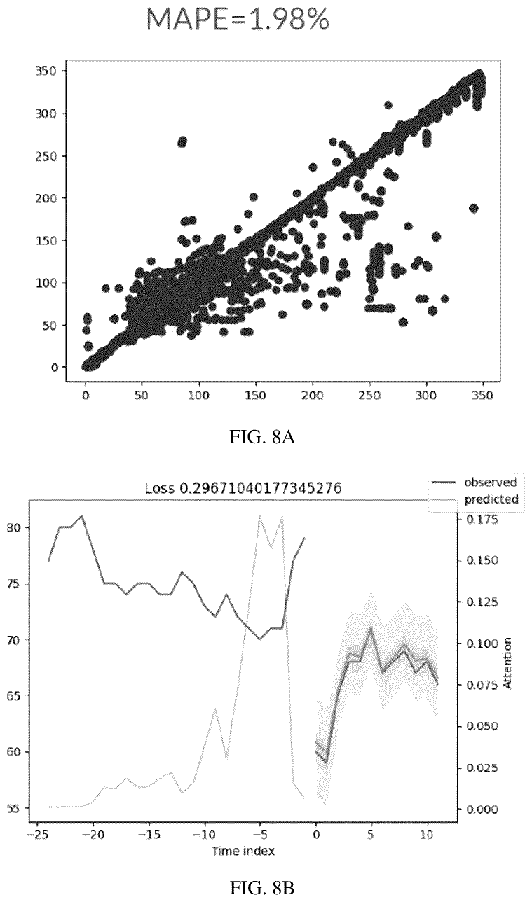

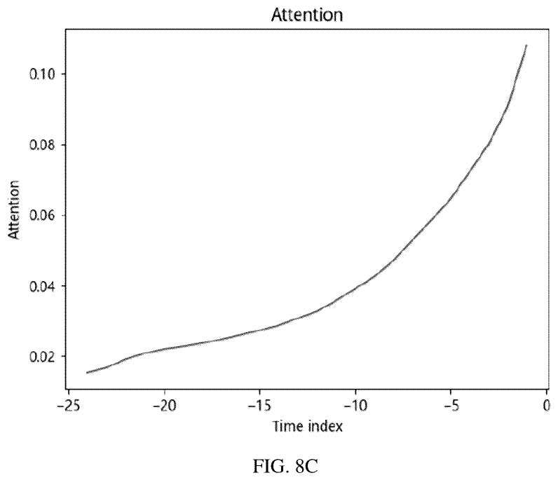

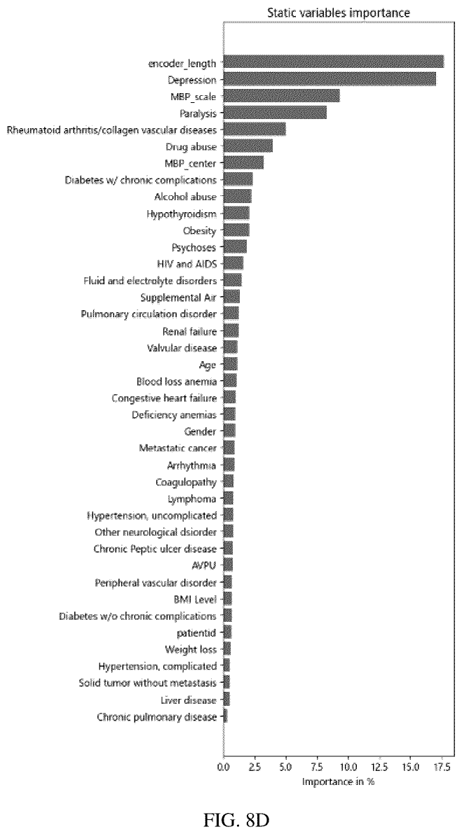

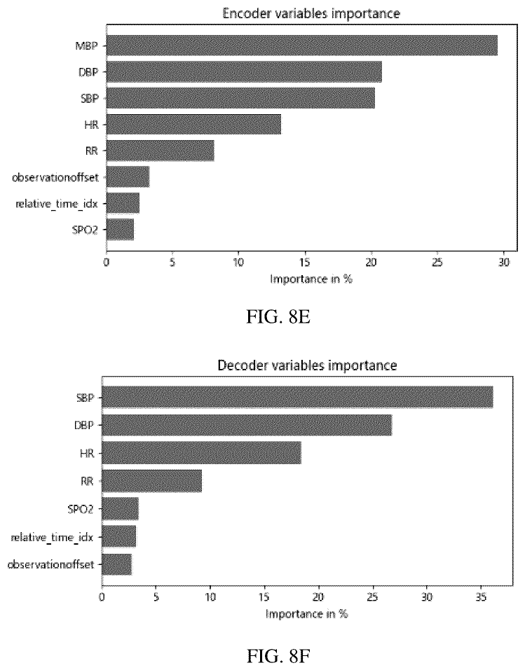

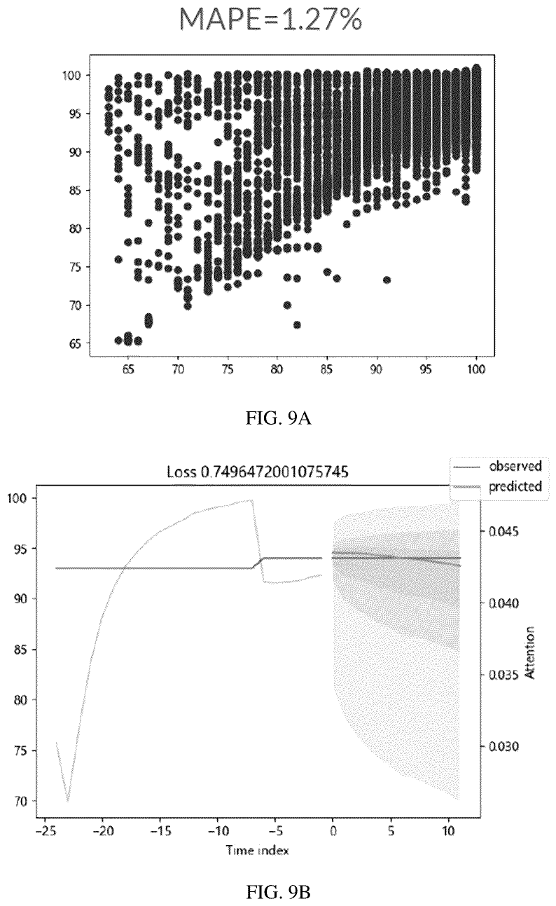

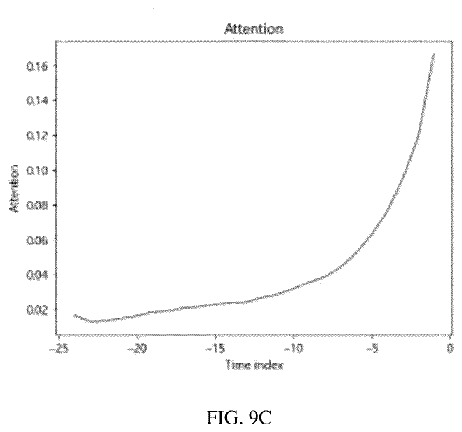

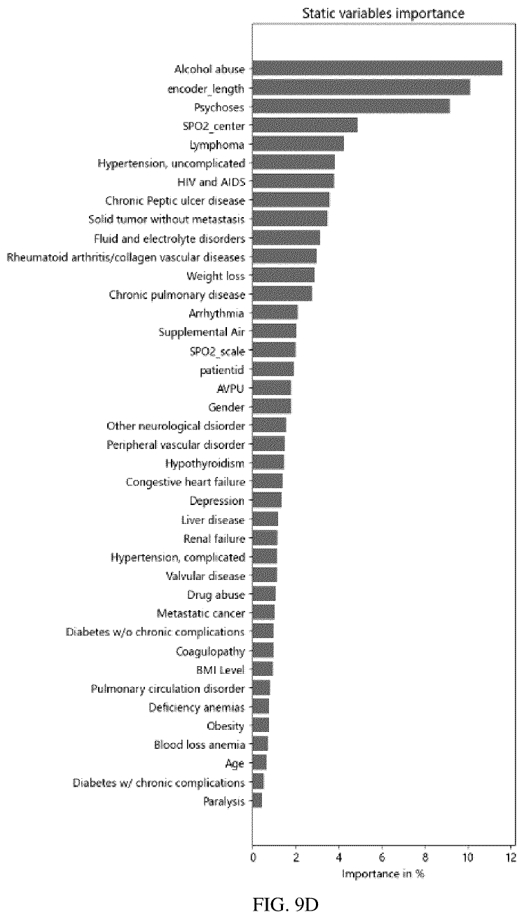

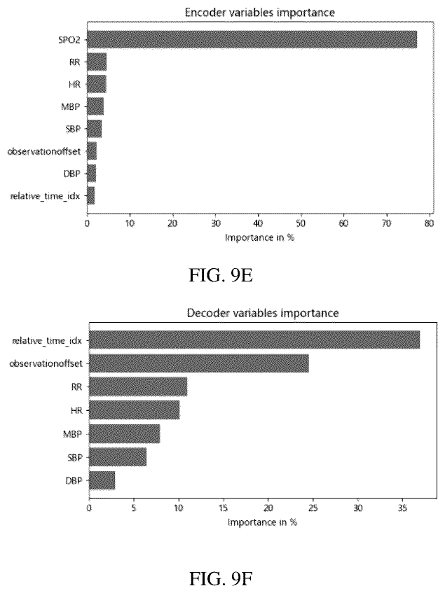

The 6 trained models are tested by the testing data. The training results for the 6 prediction models are as shown in FIGS. 4-9. The mean absolute percentage error (MAPE) of the 6 prediction models are listed below:

| Prediction | | Cardiac | Respiratory | | |
|---|---|---|---|---|---|
| Model | Control | Arrest | Failure | Shock | Total |
| HR | 4.61% | 6.16% | 4.82% | 5.02% | 4.82% |
| RR | 11.70% | 14.90% | 20.74% | 12.07% | 12.76% |
| SBP | 2.32% | 6.80% | 3.63% | 11.13% | 2.88% |
| DBP | 1.06% | 6.52% | 3.29% | 8.53% | 1.78% |
| MAP | 1.79% | 2.98% | 1.91% | 4.32% | 1.98% |
| SpO₂ | 1.07% | 2.28% | 1.30% | 1.44% | 1.27% |

(Note:
HR = heart rate;
RR = respiratory rate;
SBP = systolic pressure;
DBP = diastolic blood pressure;
MAP = mean arterial pressure;
SpO₂ = blood oxygen saturation)

The prediction models established above have learned information about patient vital signs before or in critical conditions, and thus the trained models are able to predict vital signs corresponding to various critical conditions.

### Example 3: Cardiac Arrest Prediction Model Establishment

A cardiac arrest prediction model, as described in US Patent Publication No. US2024/0032874A1, the contents of which are incorporated herein by reference, may be established to predict future risk of sudden death. Physiological monitoring data of 23,457 surviving and 3,922 dead patients obtained from public databases in the United States are used as the data for training, verification and testing in the present invention. Firstly, because the dataset in the database is imbalanced data, a total of 23,457 samples were resampled using the synthetic minority oversampling technique (SMOTE) for the death category. After that, 70% of the 23,457 survival data and 23,457 SMOTE sampled death data were used as training data (a total of 32,840 samples), 15% were used as verification data (a total of 7,037 samples), and 15% were used as test data (a total of 7,037 samples). Each training data contains 24 consecutive hours of heart rate (HR) and blood oxygen saturation (SpO₂) measurements. Each of the above training data originally contained 24 data points, and a new set of data was added between each data point by using the interpolation method, expanding the data to a total of 47 data points. The 47 data points and the survival status of the patient (0 means survival; 1 and 2 means death) constitute a complete training data set.

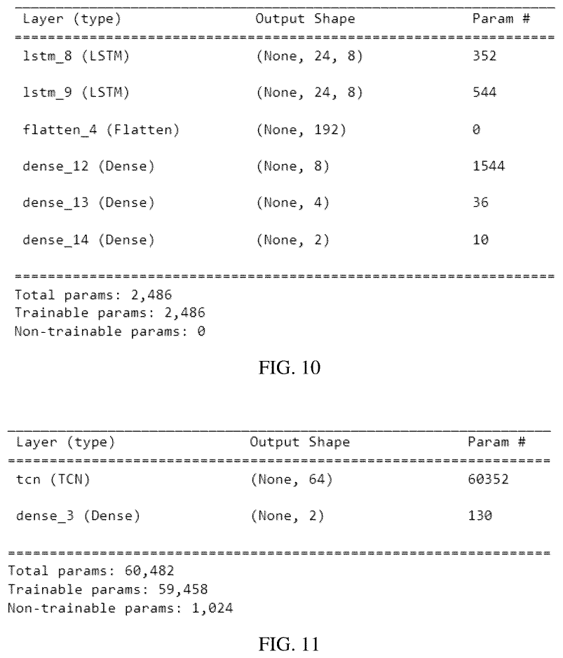

The above training data is trained with long short-term memory (LSTM) recurrent neural network (RNN). The training model employed two LSTM layers plus two fully connected layers after expansion of the LSTM layers, and then applied a Softmax layer to the last layer for training, with a total of 2,486 weights for training, and the performance of the model is monitored with the verification data, as shown in FIG. 10. The method of training optimization adopted the adaptive moment estimation (Adam) optimizer with the initial learning rate of 0.001. After the model was continuously training for 5 epochs, the learning rate decreased exponentially at a rate of 0.9 each time if the monitored numerical performance was not improve, until the value of early stopping reached 20 to stop the training. The process was optimized for at most 500 rounds. The program was executed by a Jupyter Notebook on an A100 GPU while performing the above training.

Besides LSTM, temporal convolution network (TCN) algorithm is also used for training of cardiac arrest prediction model. TCN can handle time series recognition, and the model is mainly composed of fully connected layers in addition to two convolutional layer structures combining causal convolutional layers (causal network) and dilated convolutional layers (dilated network). In the causal convolution we defined that the output value of each layer is only affected by past data, not future data. In addition, the fully connected layer ensures that the output dimension is consistent with the input dimension. Each hidden layer of the expansion convolution is consistent with the input size, and the higher of the causal convolution layer is with larger convolution window (meaning more holes). In addition to increasing the receptive field, this can also reduce the calculation. The same training data used in LSTM-RNN training is used to train TCN algorithm, and the trained model is shown in FIG. 11.

### Example 4: Combination of Vital Sign Prediction Models and Cardiac Arrest Prediction Model

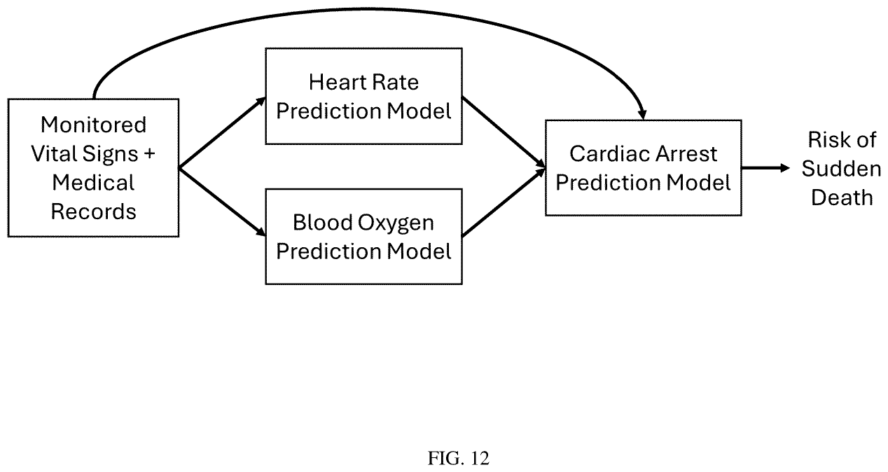

As described in US Patent Publication No. US2024/0032874A1, both the cardiac arrest prediction models with LSTM and TCN architectures have high prediction accuracy. The prediction model can predict sudden death of a patient 6 hours before occurrence of cardiac arrest. Since the vital sign prediction models only provide forecasted vital signs but not risk of critical conditions in the future, a combined model integrating both vital sign prediction models and a cardiac arrest prediction model can be established to achieve early warning, where vital signs forecasted by vital sign prediction models are used as input for cardiac arrest prediction model, as shown in FIG. 12.

The vital sign prediction models described in the above examples can take static covariates and multiple vital signs measured in previous 2 hours to forecast a target vital sign of the upcoming hour. A heart rate forecasting model can use static covariates (age, BMI level, oxygen supply condition, state of consciousness, comorbidity labels) and vital signs of previous 2 hours to forecast heart rate value of the upcoming hour. Similarly, A blood oxygen forecasting model can use the same static covariates and vital signs to forecast blood oxygen saturation level of the upcoming hour. Then 23 hours of measured heart rate and blood oxygen data, plus 1 hour of forecasted heart rate and blood oxygen data are used as input for the cardiac arrest prediction model to predict the risk of cardiac arrest. As such, the risk of cardiac arrest may be detected 1 hour earlier to provide more time for medical intervention.

Besides the above examples, many altered embodiments may also be applied to achieve the aim of early warning. For example, 6 vital sign prediction models may be used to forecast all 6 vital signs, and a cardiac arrest prediction model using all 6 vital signs to predict risk of sudden death may replace the model using only heart rate and blood oxygen data. The length of input time series for the vital sign prediction models and the cardiac arrest prediction model may also be adjusted by using different (longer or shorter) length of time series in training data, if the training results are acceptable.

Although the invention has been disclosed by preferred embodiments, it is not intended to limit the invention. The skilled artisan may make modifications without departing from the spirit and scope of the invention. Thus, the protection scope of the present invention should be defined by the claims appended below.

What is claimed is:

1. A method for training a prediction model to predict future vital signs of a subject within a predetermined future time interval, comprising training an attention-based architecture with a vital sign training dataset comprising multiple vital sign training data, each of which has a training input and a training ground truth, wherein:
the multiple vital sign training data comprise:
multiple control training data of multiple first patients without critical conditions, and
multiple emergency training data of multiple second patients with critical conditions;
the training input comprises one or more static variables and time series data of a target variable and one or more time-dependent unknown variables in an observation window; and
the training ground truth comprises time series data of the target variable in a forecast window.

2. The method of claim 1, wherein the training input further comprises time series data of one or more time-dependent known variables in the observation window and the forecast window.

3. The method of claim 1, wherein the attention-based architecture is temporal fusion transformer.

4. The method of claim 1, wherein the multiple second patients have critical conditions occur in the observation window or the forecast window.

5. The method of claim 1, wherein the critical conditions comprise cardiac arrest, shock, and/or respiratory failure.

6. The method of claim 1, wherein the one or more static variables comprise at least one of a comorbidity label, a BMI value, an oxygen supply status, and a state of consciousness.

7. The method of claim 1, wherein the target variable is selected from a group of vital signs consisting of heart rate, respiratory rate, systolic blood pressure, diastolic blood pressure, mean arterial pressure, and blood oxygen saturation.

8. The method of claim 7, wherein the one or more time-dependent unknown variables comprises the group of vital signs which are not selected as the target variable.

9. The method of claim 1, wherein the observation window comprises 24 consecutive past time points.

10. The method of claim 1, wherein the forecast window comprises 12 consecutive future time points.

11. A method to predict future risk of sudden death of a subject, comprising the steps of:
obtaining one or more static variables of the subject;
obtaining measured time series data of multiple vital signs from the subject;
based on the measured time series data and the multiple static variables, predicting forecasted time series data of heart rate by a heart rate prediction model, and predicting forecasted time series data of blood oxygen saturation by an oxygen saturation prediction model; and
based on the forecasted time series data of heart rate, the forecasted time series data of blood oxygen saturation, and/or the measured time series data, predicting a risk of sudden death of the subject by a cardiac arrest prediction model;
wherein the multiple vital signs comprise heart rate and blood oxygen saturation; and
wherein the cardiac arrest prediction model is trained by a cardiac arrest training dataset comprising multiple cardiac arrest training data, each of which consists essentially of time series data of heart rate, time series data of blood oxygen saturation, and a survival result of a patient.

12. The method of claim 11, wherein the survival result is a risk level or a probability of cardiac arrest of the subject.

13. The method of claim 11, wherein the multiple vital signs further comprise respiratory rate, systolic blood pressure, diastolic blood pressure, and mean arterial pressure.

14. The method of claim 11, wherein the one or more static variables comprise at least one of a comorbidity label, a BMI value, an oxygen supply status, and a state of consciousness.

15. The method of claim 11, wherein the forecasted time series data of heart rate and the forecasted time series data of blood oxygen saturation are predicted by using 2 hours of measured time series data and the one or more static variables to generate 1 hour of forecasted time series data.

16. The method of claim 15, wherein the cardiac arrest prediction model uses 23 hours of measured time series data and 1 hour of forecasted time series data to predict the risk of sudden death.
